(19) United States
(12) Patent Application Publication
(10) Pub. No.: US 2015/0297571 A1
(43) Pub. Date: Oct. 22, 2015

(54) Methods of Treating Hepatorenal Syndrome and Hepatic Encephalopathy with Thromboxane-A2 Receptor Antagonists

(71) Applicant: Cumberland Emerging Technologies, Inc., Nashville, TN (US)

(72) Inventors: Leo PAVLIV, Cary, NC (US); Martin Ogletree, Newton, PA (US)

(21) Appl. No.: 14/789,456

(22) Filed: Jul. 1, 2015

Related U.S. Application Data
(63) Continuation of application No. 13/738,429, filed on Apr. 10, 2013.
(60) Provisional application No. 61/364,190, filed on Jul. 14, 2010.
(60) Provisional application No. 61/364,179, filed on Jul. 14, 2010.

(51) Int. Cl. A61K 31/422

(52) U.S. Cl. CPC A61K 31/422

(57) ABSTRACT

The present invention is directed to methods of treating hepatorenal syndrome by administration of a therapeutically effective amount of a thromboxane A₂ receptor antagonist to a patient in need thereof. The present invention is also directed to methods of treating hepatic encephalopathy and cerebral edema by administration of a therapeutically effective amount of a thromboxane A₂ receptor antagonist to a patient in need thereof.

### FIELD OF THE INVENTION

The present invention is related to the use of thromboxane A₂ receptor antagonists (e.g., Ifetroban) in the treatment and/or prevention of renal diseases (e.g., hepatorenal syndrome) and hepatic renal encephalopathy; and pharmaceutical compositions for the treatment and/or prevention of renal diseases (e.g., hepatorenal syndrome) and/or hepatic renal encephalopathy, the pharmaceutical composition comprising thromboxane A₂ receptor antagonists (e.g., Ifetroban) in an effective amount to treat and/or prevent these diseases.

The present invention is also related to the field of renal diseases and, specifically to methods of preventing and/or treating hepatorenal syndrome by administration of thromboxane A₂ receptor antagonists (e.g., Ifetroban).

The present invention is further related to methods of preventing, treating, and/or improving encephalopathy and/or cerebral edema by administration of thromboxane A₂ receptor antagonists (e.g., Ifetroban).

### BACKGROUND OF THE INVENTION

### Hepatorenal Syndrome

Hepatorenal syndrome (hepatorenal syndrome) is the development of renal failure in patients with advanced chronic liver disease, occasionally fulminant hepatitis, who have portal hypertension and ascites. Estimates indicate that at least 40% of patients with cirrhosis and ascites will develop hepatorenal syndrome during the natural history of their disease.

During the 19th century, Frerichs and Flint made the original description of renal function disturbances in liver disease. They described oliguria in patients with chronic liver disease in the absence of proteinuria and linked the abnormalities in renal function to disturbances present in the systemic circulation. In the 1950s, the clinical description of hepatorenal syndrome by Sherlock, Popper, and Vessin emphasized the functional nature of the syndrome, the coexistence of systemic circulatory abnormalities, and its dismal prognosis. Further studies in the following two decades demonstrated that renal failure occurred because of vasoconstriction of the renal circulation and intense systemic arteriolar vasodilatation resulting in reduced systemic vascular resistance and arterial hypotension.

In hepatorenal syndrome, the histological appearance of the kidneys is normal, and the kidneys often resume normal function following liver transplantation. This makes hepatorenal syndrome a unique pathophysiological disorder that provides possibilities for studying the interplay between vasoconstrictor and vasodilator systems on the renal circulation.

Relevant studies include those implicating the renin-angiotensin-aldosterone system (RAAS), the sympathetic nervous system (SNS), and the role of renal prostaglandins (PGs). Strong associations have been reported between spontaneous bacterial peritonitis (SBP) and hepatorenal syndrome and the use of vasoconstrictors, including vasopressin analogues, with volume expanders in the management and prevention of hepatorenal syndrome. Although a similar syndrome may occur in acute liver failure, hepatorenal syndrome is usually described in the context of chronic liver disease. Despite some encouraging studies of new pharmacological therapies, the development of hepatorenal syndrome in people with cirrhosis portends a dismal prognosis because renal failure is usually irreversible unless liver transplantation is performed.

### Pathophysiology

The hallmark of hepatorenal syndrome is renal vasoconstriction, although the pathogenesis is not fully understood. Multiple mechanisms are probably involved and include interplay between disturbances in systemic hemodynamics, activation of vasoconstrictor systems, and a reduction in activity of the vasodilator systems. The hemodynamic pattern of patients with hepatorenal syndrome is characterized by increased cardiac output, low arterial pressure, and reduced systemic vascular resistance. Renal vasoconstriction occurs in the absence of reduced cardiac output and blood volume, which is in contrast to most clinical conditions associated with renal hypoperfusion.

Although the pattern of increased renal vascular resistance and decreased peripheral resistance is characteristic of hepatorenal syndrome, it also occurs in other conditions, such as anaphylaxis and sepsis. Doppler studies of the brachial, middle cerebral, and femoral arteries suggest that extrarenal resistance is increased in patients with hepatorenal syndrome, while the splanchnic circulation is responsible for arterial vasodilatation and reduced total systemic vascular resistance.

The renin-angiotensin-aldosterone system and sympathetic nervous system are the predominant systems responsible for renal vasoconstriction. The activity of both systems is increased in patients with cirrhosis and ascites, and this effect is magnified in hepatorenal syndrome. In contrast, an inverse relationship exists between the activity of these two systems and renal plasma flow (RPF) and the glomerular filtration rate (GFR). Endothelin is another renal vasoconstrictor present in increased concentration in hepatorenal syndrome, although its role in the pathogenesis of this syndrome has yet to be identified. Adenosine is well known for its vasodilator properties, although it acts as a vasoconstrictor in the lungs and kidneys. Elevated levels of adenosine are more common in patients with heightened activity of the renin-angiotensin-aldosterone system and may work synergistically with angiotensin II to produce renal vasoconstriction in hepatorenal syndrome. Overproduction of renal vasoconstrictor cysteinyl leukotrienes, reflected in urinary excretion of the metabolite, leukotriene E4, also been described in hepatorenal syndrome.

The vasoconstricting effect of these various systems is antagonized by local renal vasodilatory factors, the most important of which are the prostaglandins. Perhaps the strongest evidence supporting their role in renal perfusion is the marked decrease in renal plasma flow and the glomerular filtration rate when nonsteroidal anti-inflammatory drugs, medications known to sharply reduce PG levels, are administered.

Nitric oxide (NO) is another vasodilator believed to play an important role in renal perfusion. Preliminary studies, predominantly from animal experiments, demonstrate that NO production is increased in people with cirrhosis, although inhibition does not result in renal vasoconstriction due to a compensatory increase in PG synthesis. However, when both NO and prostaglandins production are inhibited, marked renal vasoconstriction develops.

These findings demonstrate that renal vasodilators play a critical role in maintaining renal perfusion, particularly in the presence of overactivity of renal vasoconstrictors. However, whether vasoconstrictor activity becomes the predominant system in hepatorenal syndrome and whether reduction in activity of the vasodilatory system contributes to this have yet to be proven.

Various theories have been proposed to explain the development of hepatorenal syndrome in cirrhosis. The two main theories are the arterial vasodilation theory and the hepatorenal reflex theory. The former theory not only describes sodium and water retention in cirrhosis, but also may be the most rational hypothesis for the development of hepatorenal syndrome. Splanchnic arteriolar vasodilatation in patients with compensated cirrhosis and portal hypertension may be mediated by several factors, the most important of which is probably nitric oxide. In the early phases of portal hypertension and compensated cirrhosis, this underfilling of the arterial bed causes a decrease in the effective arterial blood volume and results in homeostatic/reflex activation of the endogenous vasoconstrictor systems.

Activation of the renin-angiotensin-aldosterone system and sympathetic nervous system occurs early with antidiuretic hormone secretion, a later event when a more marked derangement in circulatory function is present. This results in vasoconstriction not only of the renal vessels, but also in vascular beds of the brain, muscle, spleen, and extremities. The splanchnic circulation is resistant to these effects because of the continuous production of local vasodilators such as nitric oxide.

In the early phases of portal hypertension, renal perfusion is maintained within normal or near-normal limits as the vasodilatory systems antagonize the renal effects of the vasoconstrictor systems. However, as liver disease progresses in severity, a critical level of vascular underfilling is achieved. Renal vasodilatory systems are unable to counteract the maximal activation of the endogenous vasoconstrictors and/or intrarenal vasoconstrictors, which leads to uncontrolled renal vasoconstriction. Support for this hypothesis is provided by studies in which the administration of splanchnic vasoconstrictors in combination with volume expanders results in improvement in arterial pressure, renal plasma flow, and the glomerular filtration rate.

The alternative theory proposes that renal vasoconstriction in hepatorenal syndrome is unrelated to systemic hemodynamics but is due to either a deficiency in the synthesis of a vasodilatory factor or a hepatorenal reflex that leads to renal vasoconstriction. Evidence points to the vasodilation theory as a more tangible explanation for the development of hepatorenal syndrome.

Type 1 hepatorenal syndrome is characterized by rapid and progressive renal impairment and is most commonly precipitated by spontaneous bacterial peritonitis. Type 1 hepatorenal syndrome occurs in approximately 25% of patients with spontaneous bacterial peritonitis, despite rapid resolution of the infection with antibiotics. Without treatment, median survival of patients with type 1 hepatorenal syndrome is less than 2 weeks, and virtually all patients die within 10 weeks after the onset of renal failure.

Type 2 hepatorenal syndrome is characterized by a moderate and stable reduction in the glomerular filtration rate and commonly occurs in patients with relatively preserved hepatic function. These patients are often diuretic-resistant with a median survival of 3-6 months. Although this is markedly longer than type 1 hepatorenal syndrome, it is still shorter compared to patients with cirrhosis and ascites who do not have renal failure.

### Treatment

The ideal treatment of hepatorenal syndrome is liver transplantation; however, because of the long waiting lists in the majority of transplant centers, most patients die before transplantation. An urgent need exists for effective alternative therapies to increase survival chances for patients with hepatorenal syndrome until transplantation can be performed. This is reinforced by a study that reported that patients successfully treated medically for hepatorenal syndrome before liver transplantation had posttransplantation outcome and survival comparable to that of patients who underwent transplantation without being treated for hepatorenal syndrome. Interventions that have shown some promise are drugs with vasoconstrictor effects in the splanchnic circulation and use of the transjugular intrahepatic portosystemic shunt (TIPS).

Numerous pharmacological treatments have been used to treat hepatorenal syndrome with little, if any, effect. The pharmacologic approach has shifted, however, with greater attention now focused on the role of vasoconstrictors as opposed to the initial predominant use of vasodilators. The rationale for this change is that the initial event in hepatorenal syndrome is vasodilatation of the splanchnic circulation and use of a vasoconstrictor may thus prevent homeostatic activation of endogenous vasoconstrictors. Promising results have been reported in small studies and case reports with agonists of vasopressin V1 receptors, such as ornipressin and terlipressin, which predominantly act on the splanchnic circulation.

Although only a few controlled trials have been conducted in this arena, the results so far are encouraging and suggest an increasing role for medical therapy, given the current shortage of the donor pool in the face of an ever-increasing demand for organs.

Low-dose dopamine (2-5 mcg/kg/min) is frequently prescribed to patients with renal failure in the hope that its vasodilatory properties may improve renal blood flow. Little evidence exists to support this practice; a placebo-controlled randomized trial by Bellomo and colleagues did not demonstrate any role for low-dose dopamine in early renal dysfunction. Five studies have evaluated the role of dopamine in hepatorenal syndrome, and none have reported significant changes in renal plasma flow, glomerular filtration rate, or urine output.

These studies are limited by small sample size and lack of a control arm. Nonetheless, they demonstrate that dopamine administration in patients with cirrhosis, with or without hepatorenal syndrome, does not improve renal function.

Misoprostol, a synthetic analogue of PG E1, whose use in hepatorenal syndrome was based on the observation that these patients had low urinary levels of vasodilatory prostaglandins.

Five studies have assessed the role of either parenteral or oral misoprostol in hepatorenal syndrome. None of these studies demonstrated an improvement in the glomerular filtration rate, sodium excretion, or renal function in patients with hepatorenal syndrome. Although Fevery et al demonstrated reversal of hepatorenal syndrome in 4 patients, these patients also received large doses of colloids (Fevery J, Van Cutsem E, Nevens F, Van Steenbergen W, Verberckmoes R, De Groote J. Reversal of hepatorenal syndrome in four patients by peroral misoprostol (prostaglandin E1 analogue) and albumin administration. J Hepatol. September 1990; 11(2):153-8). The likely scenario is that the massive administration of fluids played a predominant role here because Gines et al were unable to reproduce these findings with misoprostol alone.

Renal vasoconstrictor antagonists such as Saralasin, an antagonist of angiotensin II receptors, was used first in 1979 in an attempt to reverse renal vasoconstriction. Because this drug inhibited the homeostatic response to hypotension commonly observed in patients with cirrhosis, it led to worsening hypotension and deterioration in renal function. Poor results were also observed with phentolamine, an alpha-adrenergic antagonist, highlighting the importance of the SNS in maintaining renal hemodynamics in patients with hepatorenal syndrome.

A case series by Soper et al reported an improvement in the glomerular filtration rate in 3 patients with cirrhosis, ascites, and hepatorenal syndrome who received an antagonist of endothelin A receptor (BQ123) (Soper C P, Latif A B, Bending M R. Amelioration of hepatorenal syndrome with selective endothelin-A antagonist. Lancet. Jun. 29, 1996; 347(9018):1842-3). All three patients showed a dose-response improvement in inulin and para-aminohippurate excretion, renal plasma flow, and the glomerular filtration rate in the absence of changes in systemic hemodynamics. These 3 patients were not candidates for liver transplantation and subsequently died. More work is needed to explore this therapeutic approach as a possible bridge to transplantation for patients with hepatorenal syndrome.

Systemic vasoconstrictors have shown promise for the treatment of hepatorenal syndrome; they include vasopressin analogues (ornipressin, terlipressin), somatostatin analogues (octreotide), and alpha-adrenergic agonists (midodrine). In 1956, Hecker and Sherlock used norepinephrine to treat patients with cirrhosis who had hepatorenal syndrome; they were the first to describe an improvement in arterial pressure and urine output. However, no improvement was observed in biochemical parameters of renal function, and all patients subsequently died.

Octapressin, a synthetic vasopressin analogue, was first used in 1970 to treat type 1 hepatorenal syndrome. Renal plasma flow and the glomerular filtration rate improved in all patients, all of whom subsequently died from sepsis, gastrointestinal bleeding, and liver failure. Because of these discouraging results, the use of alternate vasopressin analogues, particularly ornipressin, attracted attention. Three important studies by Lenz and colleagues demonstrated that short-term use of ornipressin resulted in an improvement in circulatory function and a significant increase in renal plasma flow and the glomerular filtration rate (Lenz K, Druml W, Kleinberger G, Hortnagl H, Laggner A, Schneeweiss B, et al. Enhancement of renal function with ornipressin in a patient with decompensated cirrhosis. Gut. December 1985; 26(12): 1385-6; Lenz K, Hortnagl H, Druml W, Grimm G, Laggner A, Schneeweisz B, et al. Beneficial effect of 8-ornithin vasopressin on renal dysfunction in decompensated cirrhosis. Gut. January 1989; 30(1):90-6; and Lenz K, Hortnagl H, Druml W, Reither H, Schmid R, Schneeweiss B, et al. Ornipressin in the treatment of functional renal failure in decompensated liver cirrhosis. Effects on renal hemodynamics and atrial natriuretic factor. Gastroenterology. October 1991; 101(4):1060-7).

The combination of omipressin and albumin was subsequently tried by Guevera in patients with hepatorenal syndrome (Guevara M, Ginès P. Hepatorenal syndrome. Dig Dis. 2005; 23(1):47-55; and Guevara M, Rodés J. Hepatorenal syndrome. Int J Biochem Cell Biol. January 2005; 37(1):22-6)). This was based on data suggesting that the combination of plasma volume expansion and vasoconstrictors normalized renal sodium and water handling in patients who have cirrhosis with ascites. In this important paper, 8 patients were originally to be treated for 15 days with ornipressin and albumin. Treatment had to be discontinued in 4 patients after fewer than 9 days because of complications from ornipressin use that included ischemic colitis, tongue ischemia, and glossitis. Although a marked improvement in the serum creatinine level was observed during treatment, renal function deteriorated upon treatment withdrawal. In the remaining 4 patients, the improvement in renal plasma flow and the glomerular filtration rate was significant and was associated with a reduction in serum creatinine levels. These patients subsequently died, but no recurrence of hepatorenal syndrome was observed.

Due to the high incidence of severe adverse effects with ornipressin, the same investigators used another vasopressin analogue with fewer adverse effects, namely terlipressin. In this study, 9 patients were treated with terlipressin and albumin for 5-15 days. This was associated with a marked reduction in serum creatinine levels and improvement in mean arterial pressure. Reversal of hepatorenal syndrome was noted in 7 of 9 patients, and hepatorenal syndrome did not recur when treatment was discontinued. No adverse ischemic effects were reported, and, according to this study, terlipressin with albumin is a safe and effective treatment of hepatorenal syndrome.

Since this early study, terlipressin has become the most studied vasopressin analogue in hepatorenal syndrome. When used in conjunction with albumin, improvement in glomerular filtration rate and reduction in serum creatinine levels to below 1.5 mg/dL occur in 60-75% of patients with type 1 hepatorenal syndrome. This may take several days, and although recurrent hepatorenal syndrome after treatment discontinuation is uncommon (<15%), a repeat course of terlipressin with albumin is usually effective. Ischemic complications are also rare (<5%), but one limitation of terlipressin is its unavailability in many countries, including the United States. Under these circumstances, such agents as octreotide, albumin, and alpha-adrenergic agonists may be considered.

Gluud et al reviewed 10 randomized studies to determine whether vasoconstrictor drugs reduce mortality in patients with type 1 or type 2 hepatorenal syndrome (Gluud L L, Christensen K, Christensen E, et al. Systematic review of randomized trials on vasoconstrictor drugs for hepatorenal syndrome. Hepatology. Sep. 9, 2009). The trials, on a total of 376 patients, investigated outcomes of hepatorenal syndrome treatments using terlipressin alone or with albumin, using octreotide plus albumin, or using noradrenalin plus albumin. In their analysis, Gluud and colleagues found that administration of terlipres sin plus albumin may lead to short-term mortality reduction in patients with type 1 hepatorenal syndrome, but the authors saw no such reduction in patients with the type 2 form of the disease. Trials into octreotide and noradrenaline therapies were small and indicated neither harmful nor beneficial effects from these treatments. The authors advised that the response duration from terlipressin therapy be taken into account when treatment and the timing of liver transplantation are considered for patients with type 1 hepatorenal syndrome.

Angeli et al showed that long-term administration of midodrine (an alpha-adrenergic agonist) and octreotide improved renal function in 8 patients with type 1 hepatorenal syndrome (Angeli P, Volpin R, Gerunda G, Craighero R, Roner P, Merenda R, et al. Reversal of type 1 hepatorenal syndrome with the administration of midodrine and octreotide. Hepatology. June 1999; 29(6):1690-7). All patients also received albumin, and this approach was compared to dopamine at nonpressor doses. Not surprisingly, none of the patients treated with dopamine showed any improvement in renal function, but all 8 patients treated with midodrine, octreotide, and volume expansion had improvement in renal function. No adverse effects were reported in these patients. A study of 14 patients by Wong et al reported improvement in renal function in 10 patients. Three of these patients subsequently underwent liver transplantation.

These studies demonstrate several important points. First, vasoconstrictors play an important role in the treatment of hepatorenal syndrome, but further work is needed to identify the ideal agent and to determine if the addition of albumin is necessary. Another important conclusion of these studies is that patients may maintain relatively preserved renal function once therapy is discontinued. This suggests that if the precipitating factor, such as SBP, is not readily identified, an irreversible decline in renal function ensues.

N-acetylcysteine (NAC): In 1999, the Royal Free group reported their experience with NAC for the treatment of hepatorenal syndrome. This was based on experimental models of acute cholestasis, in which administration of NAC resulted in an improvement in renal function. Twelve patients with hepatorenal syndrome were treated with intravenous NAC, without any adverse effects, and the survival rates were 67% and 58% at 1 month and 3 months, respectively (this included 2 patients who received liver transplantation after improvement in renal function). The mechanism of action remains unknown, but this interesting study encourages further optimism for medical treatment of a condition that once carried a hopeless diagnosis in the absence of liver transplantation. Controlled studies with longer follow-up may help answer these pressing questions.

### Hepatic Encephalopathy

Hepatic encephalopathy is a syndrome observed in patients with cirrhosis. Hepatic encephalopathy is defined as a spectrum of neuropsychiatric abnormalities in patients with liver dysfunction, after exclusion of other known brain disease. Hepatic encephalopathy is characterized by personality changes, intellectual impairment, and a depressed level of consciousness. Neuropsychiatric impairment may be accompanied by decreased heart rate variability, increased blood-brain-barrier permeability and/or cerebral edema. A noted characteristic of the syndrome is diversion of portal blood into the systemic circulation through portosystemic collateral vessels. Hepatic encephalopathy is also described in patients without cirrhosis with either spontaneous or surgically created portosystemic shunts. The development of hepatic encephalopathy is explained, to some extent, by the effect of neurotoxic substances, which occurs in the setting of cirrhosis and portal hypertension.

Subtle signs of hepatic encephalopathy are observed in nearly 70% of patients with cirrhosis. Symptoms may be debilitating in a significant number of patients and are observed in 24-53% of patients who undergo portosystemic shunt surgery. Approximately 30% of patients dying of end-stage liver disease experience significant encephalopathy, approaching coma.

Hepatic encephalopathy, accompanying the acute onset of severe hepatic synthetic dysfunction, is the hallmark of fulminant hepatic failure (FHF). Symptoms of encephalopathy in fulminant hepatic failure are graded using the same scale used to assess encephalopathy symptoms in cirrhosis. The encephalopathy of cirrhosis and fulminant hepatic failure share many of the same pathogenic mechanisms. However, brain edema plays a much more prominent role in fulminant hepatic failure than in cirrhosis. The brain edema of fulminant hepatic failure is attributed to increased permeability of the blood-brain barrier, impaired osmoregulation within the brain, and increased cerebral blood flow. The resulting brain cell swelling and brain edema are potentially fatal. In contrast, brain edema is rarely reported in patients with cirrhosis.

A nomenclature has been proposed for categorizing hepatic encephalopathy. Type A hepatic encephalopathy describes encephalopathy associated with acute liver failure. Type B hepatic encephalopathy describes encephalopathy associated with portal-systemic bypass and no intrinsic hepatocellular disease. Type C hepatic encephalopathy describes encephalopathy associated with cirrhosis and portal hypertension or portal-systemic shunts. Type C hepatic encephalopathy is, in turn, subcategorized as episodic, persistent, or minimal.

### Pathophysiology

A number of theories have been proposed to explain the development of hepatic encephalopathy in patients with cirrhosis. Some investigators contend that hepatic encephalopathy is a disorder of astrocyte function. Astrocytes account for about one third of cortical volume. They play a key role in the regulation of the blood-brain barrier. They are involved in maintaining electrolyte homeostasis and in providing nutrients and neurotransmitter precursors to neurons. They also play a role in the detoxification of a number of chemicals, including ammonia.

It is theorized that neurotoxic substances, including ammonia and manganese, may gain entry into the brain in the setting of liver failure. These neurotoxic substances may then contribute to morphologic changes in astrocytes. In cirrhosis, astrocytes may undergo Alzheimer type II astrocytosis. Here, astrocytes become swollen. They may develop a large pale nucleus, a prominent nucleolus, and margination of chromatin. In fulminant hepatic failure, astrocytes may also become swollen. The changes of Alzheimer type II astrocytosis are not seen in fulminant hepatic failure. But, in contrast to cirrhosis, astrocyte swelling in fulminant hepatic failure may be so marked as to produce brain edema. This may lead to increased intracranial pressure and, potentially, brain herniation.

In the late 1990s, authors from the University of Nebraska, using epidural catheters to measure intracranial pressure (ICP), reported elevated ICP in 12 patients with advanced cirrhosis and grade 4 hepatic coma over a 6-year period (Donovan J P, Schafer D F, Shaw B W Jr, et al. Cerebral edema and increased intracranial pressure in chronic liver disease. Lancet. Mar. 7, 1998; 351(9104):719-21). Cerebral edema was reported on CT scan of the brain in 9 of the 12 patients. Several of the patients transiently responded to treatments that are typically associated with the management of cerebral edema in patients with fulminant hepatic failure. Interventions included elevation of the head of the bed, hyperventilation, intravenous mannitol, and phenobarbital-induced coma.

It was thought that patients with worsening encephalopathy should undergo head CT scan to rule out the possibility of an intracranial lesion, including hemorrhage. Certainly, cerebral edema, if discovered, should be aggressively managed. The true incidence of elevated ICP in patients with cirrhosis and severe hepatic encephalopathy remains to be determined.

Additional work focused on changes in gene expression in the brain has been conducted. The genes coding for a wide array of transport proteins may be upregulated or downregulated in cirrhosis and fulminant hepatic failure. As an example, the gene coding for the peripheral-type benzodiazepine receptor is upregulated in both cirrhosis and fulminant hepatic failure. Such alterations in gene expression may ultimately result in impaired neurotransmission.

Hepatic encephalopathy may also be thought of as a disorder that is the end result of accumulated neurotoxic substances in the brain. Putative neurotoxins include short-chain fatty acids; mercaptans; false neurotransmitters, such as tyramine, octopamine, and beta-phenylethanolamines; manganese; ammonia; and gamma-aminobutyric acid (GABA).

Hepatic encephalopathy may involve an increase in blood-brain-barrier permeability that allows blood-borne neurotoxic substances access to the central nervous system. Potential mediators of the increase in blood-brain-barrier permeability include thromboxane A2 receptor agonists, such as thromboxane A2, prostaglandin endoperoxides and F2-isoprostanes.

### Treatment

The approach to the patient with hepatic encephalopathy depends upon the severity of mental status changes and upon the certainty of the diagnosis. As an example, a patient with known cirrhosis and mild complaints of decreased concentration might be served best by an empiric trial of rifaximin or lactulose and a follow-up office visit to check its effect. However, the patient presenting in hepatic coma requires a different approach. General management recommendations include: i) excluding nonhepatic causes of altered mental function; ii) checking an arterial ammonia level in the initial assessment of a hospitalized patient with cirrhosis and with impaired mental function; iii) correcting precipitants of hepatic encephalopathy, such as metabolic disturbances, gastrointestinal bleeding, infection, and constipation; iv) avoiding medications that depress central nervous system function, especially benzodiazepines (patients with severe agitation and hepatic encephalopathy may receive haloperidol as a sedative. Treating patients who present with coexisting alcohol withdrawal and hepatic encephalopathy is particularly challenging. These patients may require therapy with benzodiazepines in conjunction with lactulose and other medical therapies for hepatic encephalopathy); v) conducting prophylactic endotracheal intubation in patients with severe encephalopathy (i.e., grade 3 or 4) who are at risk for aspiration.

Fanelli et al investigated the efficacy of using an hourglass-shaped expanded polytetrafluoroethylene (ePTFE) stent-graft to treat patients whose hepatic encephalopathy was refractory to conventional medical therapy (Fanelli F, Salvatori F M, Rabuffi P, et al. Management of refractory hepatic encephalopathy after insertion of TIPS: long-term results of shunt reduction with hourglass-shaped balloon-expandable stent-graft. AJR Am J Roentgenol. December 2009; 193(6):1696-702). In the study, 12 patients who, subsequent to receiving a transjugular intrahepatic portosystemic shunt, had developed refractory hepatic encephalopathy underwent shunt reduction with the stent-graft.

Most current therapies are designed to treat the hyperammonemia that is a hallmark of most cases of hepatic encephalopathy. These therapies include diet, cathartics, antibiotics, L-ornithine L-aspartate (LOLA), zinc, sodium benzoate, sodium phenylbutyrate, sodium phenylacetate, and L-carnitine.

While much progress has been made over the years in understanding and finding treatments for hepatorenal syndrome and hepatic encephalopathy, there still exists a need to develop new medicinal therapies based on the underlying pathophysiology of these diseases. One class of compounds that is a candidate for preventing, treating and/or improving hepatorenal syndrome and hepatic encephalopathy is the thromboxane A₂ receptor antagonists described below.

### SUMMARY OF THE INVENTION

It is an object of the present invention to provide new methods of preventing and/or treating hepatorenal syndrome.

It is another object of the present invention to provide a method for treating acute kidney injury, preventing or reversing acute renal failure, increasing renal blood flow, increasing glomerular filtration rate, increasing creatinine clearance, and decreasing serum creatinine.

It is another object of the present invention to provide a method for preventing or treating hepatic encephalopathy and coma in patients with cirrhosis.

It is another object of the present invention to provide a method of preventing or treating hepatopulmonary syndrome, cirrhotic cardiomyopathy and portpulmonary hypertension.

It is another object of the present invention to provide a method of preventing and/or treating hepatorenal syndrome by administration of a thromboxane A₂ receptor antagonist.

It is yet another object of the present invention to provide a method of preventing and/or treating hepatorenal syndrome by administration of a therapeutically effective amount of [1S-(1α,2α,3α,4α)]-2-[[3-[4-[(Pentylamino)carbonyl]-2-oxazolyl]-7-oxabicyclo[2.2.1]hept-2-yl]methyl]-benzenepropanoic acid (Ifetroban), and pharmaceutically acceptable salts thereof.

It is another object of the present invention to provide a method preventing and/or treating hepatorenal syndrome by administration of a therapeutically effective amount of [1S-(1α,2α,3α,4α)]-2-[[3-[4-[(Pentylamino)carbonyl]-2-oxazolyl]-7-oxabicyclo[2.2.1]hept-2-yl]methyl]-benzenepropanoic acid, monosodium salt (Ifetroban Sodium).

It is an object of the present invention to provide new methods of preventing, treating and/or improving hepatic encephalopathy and/or cerebral edema.

It is another object of the present invention to provide a method for preventing, treating or improving hepatic encephalopathy and coma in patients with hepatorenal syndrome.

It is another object of the present invention to provide a method of preventing or treating hepatopulmonary syndrome, cirrhotic cardiomyopathy and portpulmonary hypertension.

It is another object of the present invention to provide a method of preventing, treating and/or improving hepatic encephalopathy by administration of a thromboxane A₂ receptor antagonist.

It is yet another object of the present invention to provide a method of preventing, treating and/or improving hepatic encephalopathy by administration of a therapeutically effective amount of [1S-(1α,2α,3α,4α)]-2-[[3-[4-[(Pentylamino)carbonyl]-2-oxazolyl]-7-oxabicyclo[2.2.1]hept-2-yl]methyl]-benzenepropanoic acid (Ifetroban), and pharmaceutically acceptable salts thereof.

It is another object of the present invention to provide a preventing, treating and/or improving hepatic encephalopathy by administration of a therapeutically effective amount of [1S-(1α,2α,3α,4α)]-2-[[3-[4-[(Pentylamino)carbonyl]-2-oxazolyl]-7-oxabicyclo[2.2.1]hept-2-yl]methyl]-benzenepropanoic acid, monosodium salt (Ifetroban Sodium).

In accordance with the above objects, the present invention provides for methods of preventing, reversing or treating the above mentioned conditions (diseases) by the administration of a therapeutically effective amount of a thromboxane A₂ receptor antagonist to a patient in need thereof.

In certain embodiments, the present invention is directed to a method of treating a disease or condition in a patient in need of medicinal therapy, comprising administering to a patient in need thereof a therapeutically effective amount of a thromboxane A₂ receptor antagonist to provide a desired plasma concentration of the thromboxane A₂ receptor antagonist of about 0.1 ng/ml to about 10,000 ng/ml, wherein the desired plasma concentration results in the patient experiencing an effect selected from the group consisting of: i) an improvement in neuropsychiatric function or consciousness; ii) a decrease in astrocyte or brain swelling; iii) an increase in heart rate variability; iv) a decrease in portosystemic blood flow shunting, and v) any combination of i)-iv) to prevent or reverse hepatic encephalopathy and/or cerebral edema.

In certain other embodiments, the present invention is directed to a method of preventing or treating hepatorenal syndrome comprising administering to a patient in need thereof a therapeutically effective amount of a thromboxane A₂ receptor antagonists to a patient in need thereof.

In accordance with the above objects, the present invention provides for methods of preventing, treating and improving the above mentioned conditions (diseases) by the administration of a therapeutically effective amount of a thromboxane A₂ receptor antagonist to a patient in need thereof.

In certain embodiments, the present invention is directed to a method of treating a disease or condition in a patient in need of medicinal therapy, comprising administering to a patient in need thereof a therapeutically effective amount of a thromboxane A₂ receptor antagonist to provide a desired plasma concentration of the thromboxane A₂ receptor antagonist of about 0.1 ng/ml to about 10,000 ng/ml, wherein the desired plasma concentration results in the patient experiencing an effect selected from the group consisting of: i) an increase in renal blood flow; ii) an increase in glomerular filtration rate; iii) an increase in creatinine clearance; iv) a decrease in serum creatinine, and v) any combination of i)-iv) to prevent or reverse acute renal failure.

In certain other embodiments, the present invention is directed to a method of preventing, treating and improving hepatic encephalopathy and/or cerebral edema comprising administering to a patient in need thereof a therapeutically effective amount of a thromboxane A₂ receptor antagonists to a patient in need thereof.

### DETAILED DESCRIPTION OF THE INVENTION

In accordance with the above stated objects, it is believed that administration of a therapeutically effective amount of a thromboxane A₂ receptor antagonist to a patient in need thereof can prevent and/or treat hepatorenal syndrome and other related hepatorenal conditions.

In accordance with the above stated objects, it is also believed that administration of a therapeutically effective amount of a thromboxane A₂ receptor antagonist to a patient in need thereof can prevent, treat and/or improve hepatic encephalopathy and other related conditions associated with development of liver failure.

The phrase “therapeutically effective amount” refers to that amount of a substance that produces some desired local or systemic effect at a reasonable benefit/risk ratio applicable to any treatment. The effective amount of such substance will vary depending upon the subject and disease condition being treated, the weight and age of the subject, the severity of the disease condition, the manner of administration and the like, which can readily be determined by one of ordinary skill in the art.

The term “thromboxane A2 receptor antagonist” as used herein refers to a compound that inhibits the expression or activity of a thromboxane receptor by at least or at least about 30%, 35%, 40%, 45%, 50%, 55%, 60%, 65%, 70%, 75%, 80%, 85%, 90%, 95%, 96%, 97%, 98%, 99%, or 100% in a standard bioassay or in vivo or when used in a therapeutically effective dose. In certain embodiments, a thromboxane A2 receptor antagonist inhibits binding of thromboxane A₂ to the receptor. Thromboxane A2 receptor antagonists include competitive antagonists (i.e., antagonists that compete with an agonist for the receptor) and non-competitive antagonists. Thromboxane A2 receptor antagonists include antibodies to the receptor. The antibodies may be monoclonal. They may be human or humanized antibodies. Thromboxane A2 receptor antagonists also include thromboxane synthase inhibitors, as well as compounds that have both thromboxane A2 receptor antagonist activity and thromboxane synthase inhibitor activity.

### Thromboxane A₂ Receptor Antagonist

The discovery and development of thromboxane A₂ receptor antagonists has been an objective of many pharmaceutical companies for approximately 30 years (see, Dogne J-M, et al., Exp. Opin. Ther. Patents 11: 1663-1675 (2001)). Certain individual compounds identified by these companies, either with or without concomitant thromboxane A₂ synthase inhibitory activity, include ifetroban (BMS), ridogrel (Janssen), terbogrel (BI), UK-147535 (Pfizer), GR 32191 (Glaxo), and S-18886 (Servier). Preclinical pharmacology has established that this class of compounds has effective antithrombotic activity obtained by inhibition of the thromboxane pathway. These compounds also prevent vasoconstriction induced by thromboxane A₂ and other prostanoids that act on the thromboxane A₂ receptor within the vascular bed, and thus may be beneficial for use in preventing and/or treating hepatorenal syndrome and/or hepatic encephalopathy.

Suitable thromboxane A2 receptor antagonists for use in the present invention may include, for example, but are not limited to small molecules such as ifetroban (BMS; [1S-(1α,2α,3α,4α)]-2-[[3-[4-[(pentylamino)carbony-l]-2-oxazolyl]-7-oxabicyclo[2.2.1]hept-2 yl]methyl]benzenepropanoic acid), as well as others described in U.S. Patent Application Publication No. 2009/0012115, the disclosure of which is hereby incorporated by reference in its entirety.

Additional thromboxane A2 receptor antagonists suitable for use herein are also described in U.S. Pat. No. 4,839,384 (Ogletree); U.S. Pat. No. 5,066,480 (Ogletree, et al.); U.S. Pat. No. 5,100,889 (Misra, et al.); U.S. Pat. No. 5,312,818 (Rubin, et al.); U.S. Pat. No. 5,399,725 (Poss, et al.); and U.S. Pat. No. 6,509,348 (Ogletree), the disclosures of which are hereby incorporated by reference in their entireties. These may include, but are not limited to, interphenylene 7-oxabicyclo-heptyl substituted heterocyclic amide prostaglandin analogs as disclosed in U.S. Pat. No. 5,100,889, including:

[1S-(1α,2α,3α,4α)]-2-[[3-[4-[[(4-cyclo-hexylbutyl)amino]carbonyl]-2-oxazolyl]-7-oxabicyclo[2.2.1]-hept-2-yl]methyl]benzenepropanoic acid (SQ 33,961), or esters or salts thereof;

[1S-(1α,2α,3α,4α)]-2-[[3-[4-[[(4-chloro-phenyl)-butyl]amino]carbonyl]-2-oxazolyl]-7-oxabicyclo[2.2.1]hept-2-yl]methyl]benzenepropanoic acid or esters, or salts thereof;

[1S-(1α,2α,3α,4α)]-3-[[3-[4-[[(4-cycloh-exylbutyl)-amino]carbonyl]-2-oxazolyl]-7-oxabicyclo]2.2.1]hept-2-yl]benzene acetic acid, or esters or salts thereof;

[1S-(1α,2α,3α,4α)]-[2-[[3-[4-[[(4-cyclohexyl-butyl)amino]carbonyl]-2-oxazolyl]-7-oxabicyclo[2.2.1]hept-2-yl]methyl]phenoxy]acetic acid, or esters or salts thereof;

[1S-(1α,2α,3α,4α)]-2-[[3-[4-[[(7,7-dime-thyloctyl)-amino]carbonyl]-2-oxazolyl]-7-oxabicyclo[2.2.1]hept-2-yl]-methyl]benzenepropanoic acid, or esters or salts thereof.

7-oxabicycloheptyl substituted heterocyclic amide prostaglandin analogs as disclosed in U.S. Pat. No. 5,100,889, issued Mar. 31, 1992, including [1S-[1α,2α(Z),3α,4α)]-6-[3-[4-[[(4-cyclohexylbutyl)amino]-carbonyl]-2-oxazolyl]-7-oxabicyclo[2.2.1]hept-2-yl]-4-hexenoic acid, or esters or salts thereof;

[1S-[1α,2α(Z),3α,4α)]]-6-[3-[4-[[(4-cyclohexyl-butyl)amino]carbonyl]-2-thiazolyl]-7-oxabicyclo[2.2.1]hept-2-yl]-4-hexenoic acid, or esters or salts thereof;

[1S-[1α,2α(Z),3α,4α)]]-6-[3-[4-[[(4-cyclohexyl-butyl)methylamino]carbonyl]-2-oxazolyl]-7-oxabicyclo-[2.2.1]hept-2-yl]-4-hexenoic acid, or esters or salts thereof;

[1S-[1α,2α(Z),3α,4α)]]-6-[3-[4-[(1-pyrrolidinyl)-carbonyl]-2-oxazolyl]-7-oxabicyclo[2.2.1]hept-2-yl]-4-hexenoic acid, or esters or salts thereof;

[1S-[1α,2α(Z),3α,4α)]]-6-[3-[4-[(cyclohexylamino)-carbonyl]-2-oxazolyl]-7-oxabicyclo[2.2.1]hept-2-yl-4-hexenoic acid or esters or salts thereof;

[1S-[1α,2α(Z),3α,4α)]]-6-[3-[4-[[(2-cyclohexyl-ethyl)amino]carbonyl]-2-oxazolyl]-7-oxabicyclo[2.2.1]hept-2-yl]-4-hexenoic acid, or esters or salts thereof;

[1S-[1α,2α(Z),3α,4α)]]-6-[3-[4-[[[2-(4-chloro-phenyl)ethyl]amino]carbonyl]-2-oxazolyl]-7-oxabicyclo-[2.2.1]hept-2-yl]-4-hexenoic acid, or esters or salts thereof;

[1S-[1α,2α(Z),3α,4α)]-6-[3-[4-[[(4-chlorophenyl)-amino]carbonyl]-2-oxazolyl]-7-oxabicyclo[2.2.1]hept-2-yl]-4-hexenoic acid, or esters or salts thereof;

[1S-[1α,2α(Z),3α,4α)]]-6-[3-[4-[[[4-(4-chloro-phenyl)butyl]amino]carbonyl]-2-oxazolyl]-7-oxabicyclo-[2.2.1]hept-2-yl]-4-hexenoic acid, or esters or salts thereof;

[1S-[1α,2α(Z),3α,4α)]]-6-[3-[4.alpha.-[[-(6-cyclohexyl-hexyl)amino]carbonyl]-2-oxazolyl]-7-oxabicyclo[2.2.1]hept-2-yl]-4-hexenoic acid, or esters, or salts thereof;

[1S-[1α,2α(Z),3α,4α)]]-6-[3-[4-[[(6-cyclohexyl-hexyl)amino]carbonyl]-2-oxazolyl]-7-oxabicyclo[2.2.1]hept-2-yl]-4-hexenoic acid, or esters or salts thereof;

[1S-[1α,2α(Z),3α,4α]]-6-[3-[4-[(propylamino)-carbonyl]-2-oxazolyl]-7-oxabicyclo[2.2.1]hept-2-yl]-4-hexenoic acid, or esters or salts thereof

[1S-[1α,2α(Z),3α,4α)]]-6-[3-[4-[[(4-butylphenyl)-amino]carbonyl]-2-oxazolyl]-7-oxabicyclo[2.2.1]hept-2-yl]-4-hexenoic acid, or esters or salts thereof;

[1S-[1α,2α(Z),3α,4α)]]-6-[3-[4-[(2,3-dihydro-1H-indol-1-yl)carbonyl]-2-oxazolyl]-7-oxabicyclo(2.2.1]hept-2-yl]-4-hexenoic acid, or esters or salts thereof;

[1S-[1α,2α(Z),3α,4α)]]-6-[3-[4-[[(4-cyclohexyl-butyl)amino]carbonyl]-2-oxazolyl]-7-oxabicyclo[2.2.1]hept-2-yl]-N-(phenylsulfonyl)-4-hexenamide;

[1S-[11α,2α(Z),3α,4α)]]-6-[3-[4-[[(4-cyclohexyl-butyl)amino]carbonyl]-2-oxazolyl]-N-(ethylsulfonyl)-7-oxabicyclo[2-.2.1]hept-2-yl]-4-hexenamide;

[1S-[1α,2α(Z),3α,4α)]]-7-[3-[4-[[(4-cyclohexyl-butyl)amino]carbonyl]-2-oxazolyl]-7-oxabicyclo(2.2.1]hept-2-yl]-5-heptenoic acid, or esters or salts thereof;

[1S-[1α,2α(Z),3α,4α)]]-6-[3-[4-[[(4-cyclohexyl-butyl)amino]carbonyl]-1H-imidazol-2-yl]-7-oxabicyclo-[2.2.1]hept-2-yl]-4-hexenoic acid or esters or salts thereof;

[1S-[1α,2α,3α,4α)]-6-[3-[4-[[(7,7-dimethyloctyl)-amino]carbonyl]-2-oxazolyl]-7-oxabicyclo[2.2.1]hept-2-yl]-4-hexenoic acid, or esters or salts thereof;

[1S-[1α,2α(E),3α,4α)]]-6-[3-[4-[[(4-cyclohexyl-butyl)amino]carbonyl]-2-oxazolyl]-7-oxabicyclo[2.2.1]hept-2-yl]-4-hexenoic acid;

[1S-[1α,2α,3α,4α)]-3-[4-[[(4-(cyclohexylbutyl)-amino]carbonyl]-2-oxazolyl]-7-oxabicyclo[2.2.1]heptane-2-hexanoic acid or esters or salts thereof,

[1S-[1α,2α(Z),3α,4α)]]-6-[3-[4-[[(4-cyclohexyl-butyl)amino]carbonyl]-2-oxazolyl]-7-oxabicyclo-[2.2.1]hept-2-yl]-4-hexenoic acid, or esters or salts thereof;

7-oxabicycloheptane and 7-oxabicycloheptene compounds disclosed in U.S. Pat. No. 4,537,981 to Snitman et al, the disclosure of which is hereby incorporated by reference in its entirety, such as [1S-(1α,2α(Z),3α(1E,3S*,4R*), 4α)]]-7-[3-(3-hydroxy-4-phenyl-1-pentenyl)-7-oxabicyclo[2.2.1]hept-2-yl]-5-heptenoic acid (SQ 29,548); the 7-oxabicycloheptane substituted aminoprostaglandin analogs disclosed in U.S. Pat. No. 4,416,896 to Nakane et al, the disclosure of which is hereby incorporated by reference in its entirety, such as [1S-[1α,2α(Z),3α,4α)]]-7-[3-[[2-(phenylamino)carbonyl]-hydrazino]methyl]-7-oxabicyclo[2.2.1]hept-2-yl]-5-heptenoic acid; the 7-oxabicycloheptane substituted diamide prostaglandin analogs disclosed in U.S. Pat. No. 4,663,336 to Nakane et al, the disclosure of which is hereby incorporated by reference in its entirety, such as, [1S-[1α,2α(Z),3α,4α)]]-7-[3-[[[[(1-oxoheptyl)amino]-acetyl]amino]methyl]-7-oxabicyclo[2.2.1]hept-2-yl]-5-heptenoic acid and the corresponding tetrazole, and [1S-[1α,2α(Z),3α,4α)]]-7-[3-[[[[(4-cyclohexyl-1-oxobutyl)-amino]acetyl]amino]methyl]-7-oxabicyclo]2.2.1]hept-2-yl]-5-heptenoic acid;

7-oxabicycloheptane imidazole prostaglandin analogs as disclosed in U.S. Pat. No. 4,977,174, the disclosure of which is hereby incorporated by reference in its entirety, such as [1S-[1α,2α(Z),3α,4α)]]-6-[3-[[4-(4-cyclohexyl-1-hydroxybutyl)-1H-imidazole-1-yl]methyl]-7-oxabicyclo[2.2.1]hept-2-yl]-4-hexenoic acid or its methyl ester;

[1S-[1α, 2α(Z),3α,4α)]]-6-[3-[[4-(3-cyclohexyl-propyl)-1H-imidazol-1-yl]methyl]-7-oxabicyclo[2.2.1]hept-2-yl]-4-hexenoic acid or its methyl ester;

[1S-[1α,2α(X(Z),3α,4α)]]-6-[3-[[4-(4-cyclohexyl-1-oxobutyl)-1H-imidazol-1-yl]methyl]-7-oxabicyclo[2.2.1]hept-2-yl]-4-hexenoic acid or its methyl ester;

[1S-[1α, 2α(Z),3α,4α]]-6-[3-(1H-imidazol-1-ylmethyl)-7-oxabicyclo[2.2.1]hept-2-yl]-4-hexenoic acid or its methyl ester; or

[1S-[1α,2α(Z),3α,4α)]]-6-[3-[[4-[[(4-cyclohexyl-butyl)amino]carbonyl]-1H-imidazol-1-yl]methyl-7-oxabicyclo-[2.2.1]-hept-2-yl]-4-hexenoic acid, or its methyl ester;

The phenoxyalkyl carboxylic acids disclosed in U.S. Pat. No. 4,258,058 to Witte et al, the disclosure of which is hereby incorporated by reference in its entirety, including 4-[2-(benzenesulfamido)ethyl]phenoxy-acetic acid (BM 13,177-Boehringer Mannheim), the sulphonamidophenyl carboxylic acids disclosed in U.S. Pat. No. 4,443,477 to Witte et al, the disclosure of which is hereby incorporated by reference in its entirety, including 4-[2-(4-chlorobenzenesulfonamido)ethyl]-phenylacetic acid (BM 13,505, Boehringer Mannheim), the arylthioalkylphenyl carboxylic acids disclosed in U.S. Pat. No. 4,752,616, the disclosure of which is hereby incorporated by reference in its entirety, including 4-(3-((4-chlorophenyl)sulfonyl)propyl)benzene acetic acid.

Other examples of thromboxane A₂ receptor antagonists suitable for use herein include, but are not limited to vapiprost (which is a preferred example), (E)-5-[[[(pyridinyl)]3-(trifluoromethyl)phenyl]methylene]amino]-oxy]pentanoic acid also referred to as R68,070-Janssen Research Laboratories, 3-[1-(4-chlorophenylmethyl)-5-fluoro-3-methylindol-2-yl]-2,-2-dimethylpropanoic acid [(L-655240 Merck-Frosst) Eur. J. Pharmacol. 135(2):193, March 17, 87], 5(Z)-7-([2,4,5-cis]-4-(2-hydroxyphenyl)-2-trifl-uoromethyl-1,3-dioxan-5-yl)heptenoic acid (ICI 185282, Brit. J. Pharmacol. 90 (Proc. Suppl):228 P-Abs, March 87), 5(Z)-7-[2,2-dimethyl-4-phenyl-1,3-dioxan-cis-5-yl]heptenoic acid (ICI 159995, Brit. J. Pharmacol. 86 (Proc. Suppl):808 P-Abs., December 85), N,N′-bis[7-(3-chlorobenzeneamino-sulfony-l)-1,2,3,4-tetrahydro-isoquinolyl]disulfonylimide (SKF 88046, Pharmacologist 25(3): 116 Abs., 117 Abs, August 83), (1.alpha.(Z)-2.beta.,5.alpha.]-(+)-7-[5-[[(1,1′-biphenyl)-4-yl]-methoxy]-2-(4-morpholinyl)-3-oxocyclopentyl]-4-heptenoic acid (AH 23848-Glaxo, Circulation 72(6):1208, December 85, levallorphan allyl bromide (CM 32,191 Sanofi, Life Sci. 31 (20-21):2261, November 15, 82), (Z,2-endo-3-oxo)-7-(3-acetyl-2-bicyclo[2.2.1]heptyl-5-hepta-3Z-enoic acid, 4-phenyl-thiosemicarbazone (EP092-Univ. Edinburgh, Brit. J. Pharmacol. 84(3):595, March 85); GR 32,191 (Vapiprost)-[1R-[1.alpha.(Z), 2.beta.,3.beta.,5.alpha.]]-(+)-7-[5-([1,1′-biphenyl]-4-ylmethoxy)-3-hydroxy-2-(1-piperidinyl)cyclopentyl]-4-heptenoic acid; ICI 192,605-4(Z)-6-[(2,4,5-cis)2-(2-chlorophenyl)-4-(2-hydroxyphenyl)-1,3-dioxan-5-yl]hexenoic acid; BAY u 3405 (ramatroban)-3-[[(4-fluorophenyl)-sulfonyl]amino]-1,2,3,4-tetrahydro-9H-c-arbazole-9-propanoic acid; or ONO 3708-7-[2.alpha.,4.alpha.-(dimethylmethano)-6.beta.-(2-cyclopentyl-2.beta.-hydroxyacetami-do)-1.alpha.-cyclohexyl]-5(Z)-heptenoic acid; (.+−.)(5Z)-7-[3-endo-((phenylsulfonyl)amino]-bicyclo[2.2.1]hept-2-exo-yl]-heptenoic acid (S-1452, Shionogi domitroban, Anboxan®.); (−)6,8-difluoro-9-p-methylsulfonylben-zyl-1,2,3,4-tetrahydrocarbazol-1-yl-acetic acid (L670596, Merck) and (3-[l-(4-chlorobenzyl)-5-fluoro-3-methyl-indol-2-yl]-2,2-dimethylpropanoic acid (L655240, Merck).

The preferred thromboxane A2 receptor antagonist of the present invention is ifetroban or any pharmaceutically acceptable salts thereof.

In certain preferred embodiments the preferred thromboxane A2 receptor antagonist is ifetroban sodium (known chemically as [1S-(1α,2α,3α,4α)]-2-[[3-[4-[(Pentylamino)carbonyl]-2-oxazolyl]-7-oxabicyclo[2.2.1]hept-2-yl]methyl]-benzenepropanoic acid, monosodium salt.

### Methods of treatment

### Hepatorenal Syndrome

In certain embodiments of the present invention there is provided a method of preventing and/or treating hepatorenal syndrome by administration of a therapeutically effective amount of a thromboxane A₂ receptor antagonist to a patient in need thereof. In particular, the administration of a therapeutically effective amount of a thromboxane A₂ receptor antagonist may prevent and/or reverse acute renal failure, increase renal blood flow, increase glomerular filtration rate, increase creatinine clearance, and/or a decrease serum creatinine, thus preventing the development of and/or worsening of hepatorenal syndrome. Worsening of hepatorenal syndrome may include further decline in renal function and/or development of multi-organ failure with hepatic encephalopathy, hepatopulmonary syndrome, and/or hepatic cardiomyopathy.

The most complete characterization of a patient with acute kidney injury or hepatorenal syndrome in need of treatment would include measurement of elevated plasma concentration of F2-isoprostane, e.g., 8-iso-PGF_{2α}. Elevated plasma concentrations of F2-isoprostane for purposed of the present invention are defined as F2-isoprostane levels greater than 50 pg/mL and, exceed levels seen in patients with stable chronic liver disease or ascites who do not have hepatorenal syndrome. F2-isoprostane is a potent renal vasoconstrictor that acts via thromboxane A₂/prostaglandin endoperoxide receptor (TPr) activation which is inhibited by administration of therapeutically effective amounts of a thromboxane A₂ receptor antagonist.

Reduction of renal vasoconstriction by inhibition of A₂/prostaglandin endoperoxide receptor (TPr) activation is associated with plasma concentrations of thromboxane A₂ receptor antagonists ranging from about 0.1 ng/ml to about 10,000 ng/ml. Preferably, the plasma concentration of thromboxane A₂ receptor antagonists ranges from about 1 ng/ml to about 1,000 ng/ml.

When the thromboxane A₂ receptor antagonists is ifetroban, the desired plasma concentration for providing an inhibitory effect of A₂/prostaglandin endoperoxide receptor (TPr) activation, and thus a reduction of vasoconstriction should be greater than about 10 ng/mL (ifetroban free acid). Some inhibitory effects of thromboxane A₂ receptor antagonist, e.g., ifetroban, may be seen at concentrations of greater than about 1 ng/mL.

The dose administered must be carefully adjusted according to age, weight and condition of the patient, as well as the route of administration, dosage form and regimen and the desired result.

However, in order to obtain the desired plasma concentration of thromboxane A₂ receptor antagonists, daily doses of the thromboxane A₂ receptor antagonists ranging from about 0.1 mg to about 5000 mg should be administered. Preferably, the daily dose of thromboxane A₂ receptor antagonists ranges from about 1 mg to about 1000 mg; about 10 mg to about 1000 mg; about 50 mg to about 500 mg; about 100 mg to about 500 mg; about 200 mg to about 500 mg; about 300 mg to about 500 mg; and about 400 mg to about 500 mg per day.

In certain preferred embodiments, a daily dose of ifetroban sodium from about 10 mg to about 250 mg (ifetroban free acid amounts) will produce effective plasma levels of ifetroban free acid.

### Hepatic Encephalopathy

In certain embodiments of the present invention there is provided a method of preventing, treating and/or improving hepatic encephalopathy by administration of a therapeutically effective amount of a thromboxane A₂ receptor antagonist to a patient in need thereof. In particular, the administration of a therapeutically effective amount of a thromboxane A₂ receptor antagonist may prevent and/or reverse an increase in blood-brain-barrier permeability, development of cerebral edema and/or brain or astrocyte swelling, thus preventing the development of and/or worsening of hepatic encephalopathy. Worsening of hepatic encephalopathy may be associated with decline in renal function and/or development of multi-organ failure with hepatopulmonary syndrome, and/or hepatic cardiomyopathy.

The most complete characterization of a patient with hepatic encephalopathy in need of treatment would include measurement of elevated plasma concentration of F2-isoprostane, e.g., 8-iso-PGF_{2α}. Elevated plasma concentrations of F2-isoprostane for purposes of the present invention are defined as F2-isoprostane levels greater than 50 pg/mL and exceed levels seen in patients with stable chronic liver disease or ascites. F2-isoprostane is a potent cerebral microvascular activator that acts via thromboxane A₂/prostaglandin endoperoxide receptor (TPr) activation which is inhibited by administration of therapeutically effective amounts of a thromboxane A₂ receptor antagonist.

Reduction of cerebral microvascular activation by inhibition of A₂/prostaglandin endoperoxide receptor (TPr) activation is associated with plasma concentrations of thromboxane A₂ receptor antagonists ranging from about 0.1 ng/ml to about 10,000 ng/ml. Preferably, the plasma concentration of thromboxane A₂ receptor antagonists ranges from about 1 ng/ml to about 1,000 ng/ml.

When the thromboxane A₂ receptor antagonist is ifetroban, the desired plasma concentration for providing an inhibitory effect of A₂/prostaglandin endoperoxide receptor (TPr) activation, and thus a reduction of cerebral microvascular activation should be greater than about 10 ng/mL (ifetroban free acid). Some inhibitory effects of thromboxane A₂ receptor antagonist, e.g., ifetroban, may be seen at concentrations of greater than about 1 ng/mL.

The dose administered must be carefully adjusted according to age, weight and condition of the patient, as well as the route of administration, dosage form and regimen and the desired result.

However, in order to obtain the desired plasma concentration of thromboxane A₂ receptor antagonists, daily doses of the thromboxane A₂ receptor antagonists ranging from about 0.1 mg to about 5000 mg should be administered. Preferably, the daily dose of thromboxane A₂ receptor antagonists ranges from about 1 mg to about 1000 mg; about 10 mg to about 1000 mg; about 50 mg to about 500 mg; about 100 mg to about 500 mg; about 200 mg to about 500 mg; about 300 mg to about 500 mg; and about 400 mg to about 500 mg per day.

In certain preferred embodiments, a daily dose of ifetroban sodium from about 10 mg to about 250 mg (ifetroban free acid amounts) will produce effective plasma levels of ifetroban free acid.

### Pharmaceutical Compositions

The thromboxane A₂ receptor antagonists of the present invention may be administered by any pharmaceutically effective route. For example, the thromboxane A₂ receptor antagonists may be formulated in a manner such that they can be administered orally, intranasally, rectally, vaginally, sublingually, buccally, parenterally, or transdermally, and, thus, be formulated accordingly.

In certain embodiments, the thromboxane A₂ receptor antagonists may be formulated in a pharmaceutically acceptable oral dosage form. Oral dosage forms may include, but are not limited to, oral solid dosage forms and oral liquid dosage forms.

Oral solid dosage forms may include, but are not limited to, tablets, capsules, caplets, powders, pellets, multiparticulates, beads, spheres and any combinations thereof. These oral solid dosage forms may be formulated as immediate release, controlled release, sustained (extended) release or modified release formulations.

The oral solid dosage forms of the present invention may also contain pharmaceutically acceptable excipients such as fillers, diluents, lubricants, surfactants, glidants, binders, dispersing agents, suspending agents, disintegrants, viscosity-increasing agents, film-forming agents, granulation aid, flavoring agents, sweetener, coating agents, solubilizing agents, and combinations thereof.

Depending on the desired release profile, the oral solid dosage forms of the present invention may contain a suitable amount of controlled-release agents, extended-release agents, modified-release agents.

Oral liquid dosage forms include, but are not limited to, solutions, emulsions, suspensions, and syrups. These oral liquid dosage forms may be formulated with any pharmaceutically acceptable excipient known to those of skill in the art for the preparation of liquid dosage forms. For example, water, glycerin, simple syrup, alcohol and combinations thereof.

In certain embodiments of the present invention, the thromboxane A₂ receptor antagonists may be formulated into a dosage form suitable for parenteral use. For example, the dosage form may be a lyophilized powder, a solution, suspension (e.g., depot suspension).

In other embodiments, the thromboxane A₂ receptor antagonists may be formulated into a topical dosage form such as, but not limited to, a patch, a gel, a paste, a cream, an emulsion, liniment, balm, lotion, and ointment.

### DETAILED DESCRIPTION OF THE PREFERRED EMBODIMENTS

The following examples are not meant to be limiting and represent certain embodiments of the present invention.

### Example 1

In this example, ifetroban sodium tablets are prepared with the following ingredients listed in Table 1:

TABLE 1

| Ingredients | Percent by weight |
|---|---|
| Na salt of Ifetroban | 35 |
| Mannitol | 50 |
| Microcrystalline Cellulose | 8 |
| Crospovidone | 3.0 |
| Magnesium Oxide | 2.0 |
| Magnesium Stearate | 1.5 |
| Colloidal Silica | 0.3 |

The sodium salt of ifetroban, magnesium oxide, mannitol, microcrystalline cellulose, and crospovidone is mixed together for about 2 to about 10 minutes employing a suitable mixer. The resulting mixture is passed through a #12 to #40 mesh size screen. Thereafter, magnesium stearate and colloidal silica are added and mixing is continued for about 1 to about 3 minutes.

The resulting homogeneous mixture is then compressed into tablets each containing 35 mg, ifetroban sodium salt.

### Example II

In this example, 1000 tablets each containing 400 mg of Ifetroban sodium are produced from the following ingredients listed in Table 2:

TABLE 2

| Ingredients | Amount | |
|---|---|---|
| Na salt of Ifetroban | 400 | gm |
| Corn Starch | 50 | g |
| Gelatin | 7.5 | g |
| Microcrystalline Cellulose (Avicel) | 25 | g |
| Magnesium Stearate | 2.5 | g |

### Example III

In this example. An injectable solution of ifetroban sodium is prepared for intravenous use with the following ingredients listed in Table 3:

TABLE 3

| Ingredients | Amount | |
|---|---|---|
| Ifetroban Sodium | 2500 | mg |
| Methyl Paraben | 5 | mg |
| Propyl Paraben | 1 | mg |
| Sodium Chloride | 25,000 | mg |
| Water for injection q.s. | 5 | liter |

The sodium salt of ifetroban, preservatives and sodium chloride are dissolved in 3 liters of water for injection and then the volume is brought up to 5 liters. The solution is filtered through a sterile filter and aseptically filled into pre-sterilized vials which are then closed with pre-sterilized rubber closures. Each vial contains a concentration of 75 mg of active ingredient per 150 ml of solution.

### Example IV

### Ifetroban Pharmacokinetic and Pharmacodynamic Safety Study

The plan to develop ifetroban to treat hepatorenal syndrome (HRS) is based on the hypothesis that high levels of liver-derived isoprostanes mediate renal vasospasm via thromboxane receptor (TPr) activation, and the TPr antagonist, ifetroban, will block isoprostane-dependent renal vasoconstriction, improve renal blood flow and reverse HRS. Development of ifetroban for this indication requires first the study of safety and pharmacokinetics of ifetroban in HRS patients. At the same time, evidence is sought that ifetroban can increase renal blood flow and be beneficial as HRS treatment.

The following clinical study is a Phase II, prospective, double-blind, placebo controlled multi-center study that will evaluate the safety, tolerability, pharmacokinetics and pharmacodynamics of ifetroban administered as multiple daily oral doses in hepatorenal syndrome type 1 patients. Hepatorenal syndrome type 1 patients will be assigned according to a dose escalation randomization schedule. Escalation to the higher doses will be contingent upon the safety and tolerability of the preceding dose. Patients may receive study drug for a maximum of 14 days but will be discontinued from the study earlier for treatment failure (defined as serum creatinine (SCr) level≧2× the baseline value after day 7, dialysis, or death) or liver transplantation. Patients who achieve treatment success may be discontinued or continue on therapy at the investigator's discretion until the maximum of 14 days. If judged by the investigator to be potentially beneficial, patients who demonstrate at least a partial response during the initial 14-day treatment course and then develop recurrence of hepatorenal syndrome type 1 during the follow-up period will be eligible to be retreated with the highest well-tolerated dose of ifetroban for up to an additional 14 days.

The primary pharmacodynamic measure of renal function will be creatinine clearance, which should increase if renal function improves.

Secondary outcomes will be evaluated, including changes in SCr and BUN levels, change in urine output and estimated GFR, and dialysis requirements.

Thirty-six (36) adult male or female (≧18 years of age) hepatorenal syndrome type 1 patients will be enrolled and assigned according to a randomization schedule to three (3) groups of twelve (12) patients each to receive on days 1 and 2 either placebo, low-dose ifetroban or high-dose ifetroban as daily oral doses.

Ifetroban study drug will be provided as look-alike capsules containing 0, 10, 50 or 125 mg of ifetroban sodium measured as free acid equivalents.

Placebo will be supplied in look-alike capsules containing formulation with no ifetroban.

Three (3) groups of twelve (12) patients each will receive on days 1 and 2 either placebo, 10 mg ifetroban or 50 mg ifetroban as daily oral doses. On days 3 and 4, daily oral doses will be increased to 50 mg ifetroban, 125 mg ifetroban and 250 mg ifetroban, respectively. On days 5 and 6, daily oral doses in all groups will be 250 mg ifetroban. Treatment will continue with daily doses of the highest well-tolerated dose for the duration of hospitalization or through day 14.

### Example V

### Ifetroban Pharmacokinetic and Pharmacodynamic Safety Study

The plan to develop ifetroban to treat hepatic encephalopathy is based on the hypothesis that high levels of liver-derived isoprostanes mediate microvascular constriction and permeability via thromboxane receptor (TPr) activation, and the TPr antagonist, ifetroban, will block isoprostane-dependent microvascular constriction and permeability, normalize cerebral blood flow and reverse or prevent progression of hepatic encephalopathy. Development of ifetroban for this indication requires first the study of safety and pharmacokinetics of ifetroban in hepatic encephalopathy patients. At the same time, evidence is sought that ifetroban can improve indices of hepatic encephalopathy, such as neuropsychiatric function and heart rate variability. and be beneficial as hepatic encephalopathy treatment.

The following clinical study is a Phase II, prospective, double-blind, placebo controlled multi-center study that will evaluate the safety, tolerability, pharmacokinetics and pharmacodynamics of ifetroban administered as one or more daily oral doses in hepatic encephalopathy patients. Hepatic encephalopathy patients will be assigned according to a dose escalation randomization schedule. Escalation to the higher doses will be contingent upon the safety and tolerability of the preceding dose. Patients may receive study drug for a maximum of 14 days but will be discontinued from the study earlier for treatment failure (defined as worsening of encephalopathy, development of coma, or death) or liver transplantation. Patients who achieve treatment success may be discontinued or continue on therapy at the investigator's discretion until the maximum of 14 days. If judged by the investigator to be potentially beneficial, patients who demonstrate at least a partial response during the initial 14-day treatment course and then develop recurrence of hepatic encephalopathy during the follow-up period will be eligible to be retreated with the highest well-tolerated dose of ifetroban for up to an additional 14 days.

The primary pharmacodynamic measure of hepatic encephalopathy will be heart rate variability which should increase if hepatic encephalopathy improves.

Secondary outcomes will be evaluated, including asterixis, which should moderate if hepatic encephalopathy improves, and changes in serum creatinine which should decrease if renal function improves.

Thirty-six (36) adult male or female (≧18 years of age) patients will be enrolled and assigned according to a randomization schedule to three (3) groups of twelve (12) patients each to receive on days 1 and 2 either placebo, low-dose ifetroban or high-dose ifetroban as daily oral doses.

Ifetroban study drug will be provided as look-alike capsules containing 0, 10, 50 or 125 mg of ifetroban sodium measured as free acid equivalents.

Placebo will be supplied in look-alike capsules containing formulation with no ifetroban.

Three (3) groups of twelve (12) patients each will receive on days 1 and 2 either placebo, 10 mg ifetroban or 50 mg ifetroban as daily oral doses. On days 3 and 4, daily oral doses will be increased to 50 mg ifetroban, 125 mg ifetroban and 250 mg ifetroban, respectively. On days 5 and 6, daily oral doses in all groups will be 250 mg ifetroban. Treatment will continue with daily doses of the highest well-tolerated dose for the duration of hospitalization or through day 14.

In the preceding specification, the invention has been described with reference to specific exemplary embodiments and examples thereof. It will, however, be evident that various modifications and changes may be made thereto without departing from the broader spirit and scope of the invention as set forth in the claims that follow. The specification and drawings are accordingly to be regarded in an illustrative manner rather than a restrictive sense.

1. A method of treating a disease or condition in a patient in need of medicinal therapy, comprising:
administering to a patient in need thereof a therapeutically effective amount of a thromboxane A₂ receptor antagonist consisting of [1S-(1α,2α,3α,4α)]-2-[[3-[4-[(Pentylamino)carbonyl]-2-oxazolyl]-7-oxabicyclo[2.2.1]hept-2-yl]methyl]-benzenepropanoic acid (ifetroban), and pharmaceutically acceptable salts thereof to provide a desired ifetroban plasma concentration of about 1 ng/ml to about 1,000 ng/ml, wherein the desired plasma concentration results in the patient experiencing an effect selected from the group consisting of:
i) an increase in renal blood flow;
ii) an increase in glomerular filtration rate;
iii) an increase in creatinine clearance;
iv) a decrease in serum creatinine, and
v) any combination of i)-iv) to prevent or reverse acute renal failure.

2. The method of claim 1, wherein the disease or condition is hepatorenal syndrome type I or type II.

3. The method of claim 1, wherein the thromboxane A₂ receptor antagonist is administered orally, intranasally, rectally, vaginally, sublingually, buccally, parenterally, or transdermally,

4. The method of claim 3, wherein the ifetroban is administered orally.

5. The method of claim 3, wherein the ifetroban is administered parenterally.

6. (canceled)

7. The method of claim 1, wherein the thromboxane A₂ receptor antagonist is [1S-(1α,2α,3α,4α)]-2-[[3-[4-[(Pentylamino)carbonyl]-2-oxazolyl]-7-oxabicyclo[2.2.1]hept-2-yl]methyl]-benzenepropanoic acid, monosodium salt (Ifetroban Sodium).

8. A method of preventing or treating hepatorenal syndrome comprising:
administering to a patient in need thereof a therapeutically effective amount of [1S-(1α,2α,3α,4α)]-2-[[3-[4-[(Pentylamino)carbonyl]-2-oxazolyl]-7-oxabicyclo[2.2.1]hept-2-yl]methyl]-benzenepropanoic acid (Ifetroban), and pharmaceutically acceptable salts thereof to a patient in need thereof.

9. The method of claim 8, wherein the hepatorenal syndrome is type I or type II.

10. (canceled)

11. The method of claim 10, wherein the thromboxane A₂ receptor antagonist is administered orally.

12. The method of claim 10, wherein the thromboxane A₂ receptor antagonist is administered parenterally.

13. (canceled)

14. The method of claim 8, wherein the thromboxane A₂ receptor antagonist is [1S-(1α,2α,3α,4α)]-2-[[3-[4-[(Pentylamino)carbonyl]-2-oxazolyl]-7-oxabicyclo[2.2.1]hept-2-yl]methyl]-benzenepropanoic acid, monosodium salt (Ifetroban Sodium).

15-31. (canceled)

32. The method of claim 1, wherein the administration of ifetroban to the patient results in an increase in renal blood flow.

33. The method of claim 1, wherein the administration of ifetroban to the patient results in an increase in glomerular filtration rate.

34. The method of claim 1, wherein the administration of ifetroban to the patient results in a decrease in serum creatinine.

35. The method of claim 1, wherein the ifetroban is orally administered as ifetroban sodium to the patient in an amount from about 10 mg to about 400 mg, per day.

36. The method of claim 35, further comprising administering to the patient a daily dose of ifetroban sodium from about 10 mg to about 50 mg on day 1 and 2, from about 50 mg to about 250 mg ifetroban sodium on days 3 and 4, and at least about 250 mg through day 14 of treatment.

37. The method of claim 1, wherein the amount of ifetroban administered is from about 10 mg to about 500 mg, per day.
